(19) United States
(12) Patent Application Publication
(10) Pub. No.: US 2018/0289919 A1
(43) Pub. Date: Oct. 11, 2018

(54) BRAINWAVE REGULATION DEVICE AND BRAINWAVE REGULATION METHOD

(71) Applicant: SUNWISE OPTICS CO., LTD, Taipei (TW)

(72) Inventors: CHENG-CHANG CHEN, Taipei (TW); YU-HSIANG CHEN, Taipei (TW)

(21) Appl. No.: 15/570,727

(22) PCT Filed: May 13, 2015
(86) PCT No.: PCT/CN2015/078859

§ 371 Date: Oct. 30, 2017

(51) Int. Cl. A61M 21/02; G06F 3/01; H04R 1/10; A61B 5/048; A61B 5/00

(52) U.S. Cl. CPC A61M 21/02; G06F 3/015; H04R 1/10; A61M 2021/0033; A61B 5/0077; H04R 2460/13; A61B 5/048

(57) ABSTRACT

A brainwave regulation device adapted to transmit signals to or receive signals from a control device. The brainwave regulation device includes a head-wearable element, a control module, at least one brainwave sensor and at least one ear bone oscillator. The control module is disposed on the head-wearable element and adapted to transmits signals to or receive signals from the control device. The at least one brainwave sensor is connected to the head-wearable element and adapted to transmit signals to or receive signals from the control module and transmit the sensed brainwave signal to the control device through the control module. The at least one ear bone oscillator is connected to the head-wearable element, and adapted to transmit signals to or receive signals from the control module and receive control signals from the control device through the control module to generate oscillation. The present invention also provides a brainwave regulation method adapted for use with a brainwave regulation device and for brainwave regulation.

### FIELD OF THE INVENTION

The present invention relates to a brainwave regulation device, and more particularly to a brainwave regulation device and a brainwave regulation method adapted to use with the brainwave regulation device for brainwave regulation.

### BACKGROUND OF THE INVENTION

Brainwave is an action potential created by cerebral cortex. The brainwave may be divided into a delta wave (δ wave) whose frequency ranges from 0.1 Hz to 3 Hz, a theta wave (θ wave) whose frequency ranges from 4 Hz to 7 Hz, an alpha wave (α wave) whose frequency ranges from 8 Hz to 15 Hz, a beta wave (β wave) whose frequency ranges from 12.5 Hz to 28 Hz and a gamma wave (γ wave) whose frequency ranges from 25 Hz to 100 Hz according to electroencephalogram. When the brainwave frequency is within the delta wave range, the brain is in a deep sleep state. When the brainwave frequency is within the alpha wave range, the brain is in a relaxed state. When the brainwave frequency is within the beta wave range and gamma wave range, the brain is in an awaken state.

It is now known that brain is stimulated and the brainwave is regulated through listening to music or songs. Music or songs of different frequencies can be used to adjust a user's brainwaves, for example, when the user's brainwave frequency is regulated to within the alpha wave frequency range, the user can be in a relaxed state. However because only music or songs of specific frequency can adjust the user's brainwave to a desired frequency range, when the user needs to use music or songs to regulate his/her brainwave, he/she needs to continuously choose preferred music or songs to regulate the brainwave, so that he/she cannot concentrate. In addition, because the user usually wear a headset to listen to music or songs, it is also easy to make ears uncomfortable for wearing the headset for a long time.

### SUMMARY OF THE INVENTION

One objective of the present invention is to provide a brainwave regulation device capable of achieving the effect of having a comfortable sleep or good spirit by oscillating ear bones.

Another objective of the present invention is to provide a brainwave regulation method capable of achieving the effect of having a comfortable sleep or good spirit by generating appropriate oscillating frequency.

To achieve advantages above, the invention provides a brainwave regulation device adapted to transmit signals to or receive signals from a control device. The brainwave regulation device includes a head-wearable element, a control module, at least one brainwave sensor and at least one ear bone oscillator. The control module is disposed on the head-wearable element and adapted to transmits signals to or receive signals from the control device. The at least one brainwave sensor is connected to the head-wearable element and adapted to transmit signals to or receive signals from the control module and transmit the sensed brainwave signal to the control device through the control module. The at least one ear bone oscillator is connected to the head-wearable element and adapted to transmit signals to or receive signals from the control module and receive control signals from the control device through the control module to generate oscillation.

In an embodiment of the invention, the brainwave regulation device further includes an identification element disposed on the head-wearable element.

In an embodiment of the invention, the identification element includes a radio-frequency element or an identification code.

In an embodiment of the invention, the brainwave regulation device further includes an image capturing device adapted to capture an image of a face or an iris of a user and transmit the image to the control device through the control module.

In an embodiment of the invention, the signals transmitted between the control device and the control module include a blue tooth signal, a wireless network signal, a near field communication signal, a ZigBee signal, an ultra-wideband signal or a light fidelity signal.

In an embodiment of the invention, the head-wearable element includes two head wearable members, the control module includes two control elements, wherein the at least one brainwave sensor and one of the control elements are disposed on one of the head wearable member, and the at least one ear bone oscillator and the other of the control elements are disposed on the other one of the head wearable members.

The present invention also provides a brainwave regulation method adapted to use with a brainwave regulation device for brainwave regulation. The brainwave regulation method includes a head wearable element, a control module, at least one brainwave sensor and at least one era bone oscillator. The brainwave regulation method includes steps: detecting a brainwave frequency by the at least one brainwave sensor; and generating, according to the brainwave frequency, at least one control signal to drive the era bone oscillator to oscillate, wherein the at least one control signal includes an oscillating frequency signal and a time.

In an embodiment of the invention, the method of generating the oscillating frequency includes steps of: comparing a difference between the brainwave frequency and a target frequency to generate an oscillating frequency so as to regulate the brainwave frequency to approach the target frequency and change a state of a user. When the oscillating frequency is lower than the brainwave frequency, the user is regulated from an awaken state to a relaxed state or from a relaxed state to a deep sleep state. When the oscillating frequency is higher than the brainwave frequency, the user is regulated from a deep sleep state to a relaxed state or from a relaxed state to an awaken state.

In an embodiment of the invention, the target frequency ranges from 0.1 Hz to 100 Hz.

In an embodiment of the invention, the target frequency is represented by G, the brainwave frequency is represented by D, the oscillating frequency is represented by S, and |G-S|≤|D-G|, |G-S|=(1/n)|D-G|, wherein n is greater than 1 and n includes a constant, a linear function or a non-linear function.

The brainwave regulation device of the present invention oscillates the user's ear bone through the era bone oscillator disposed on the head wearable element to further regulate the user's brainwave frequency. The user may have a comfortable sleep or good spirit without wearing headsets. In addition, the brainwave regulation method of the invention generates appropriate oscillating frequency and creates vibration through the ear bone oscillator to further regulate the user's brainwave frequency. Therefore, the user may have a comfortable sleep and good spirit without adjusting the music or songs to which the user is listening.

The description above is merely a summary of technical solutions of the present invention to more clearly understand the structure of the present invention and practice the invention according to the specification. A detailed description is given in the following embodiments with reference to the accompanying drawings.

### BRIEF DESCRIPTION OF THE DRAWINGS

FIG. 1 is a schematic diagram of an embodiment of a brainwave regulation device of the present invention.

FIG. 2 is a block diagram of an embodiment of a brainwave regulation device and a control device of the present invention.

FIG. 3A is a schematic diagram of another embodiment of a brainwave regulation device of the present invention.

FIG. 3B is a schematic diagram of still another embodiment of a brainwave regulation device of the present invention.

FIG. 4 is a schematic diagram of still another embodiment of a brainwave regulation device and a control device of the present invention.

FIG. 5 is a schematic diagram of another embodiment of a brainwave regulation device of the present invention.

FIG. 6 is a block diagram of another embodiment of a brainwave regulation device and a control device of the present invention.

FIG. 7 is a block diagram of an embodiment of a brainwave regulation method of the present invention.

### DETAILED DESCRIPTION OF PREFERRED EMBODIMENTS

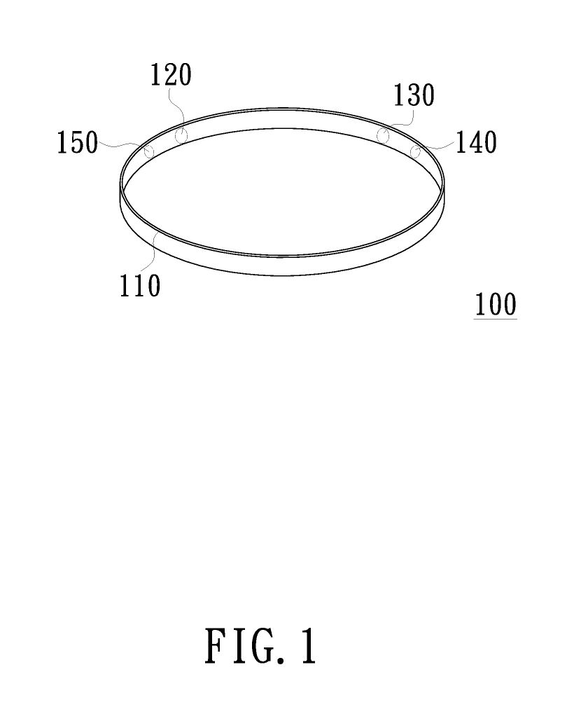

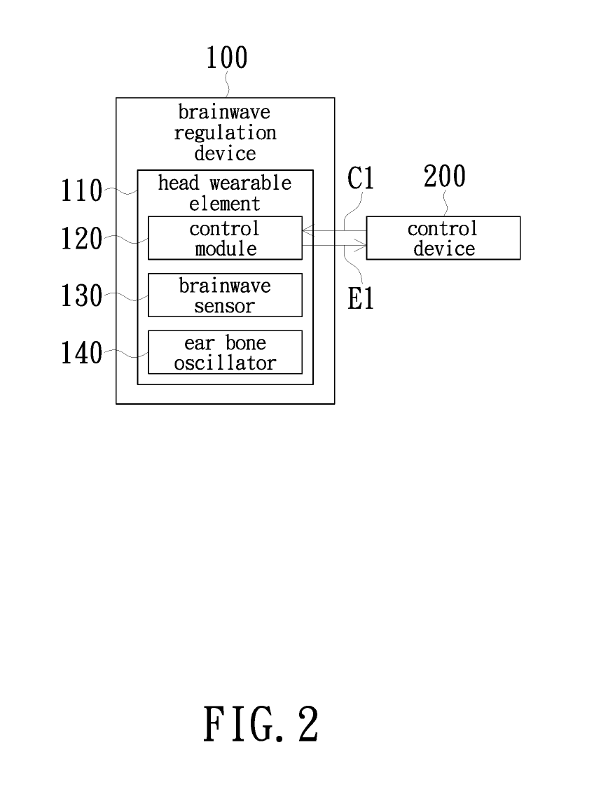

FIG. 1 is a schematic diagram of an embodiment of a brainwave regulation device of the present invention. FIG. 2 is a block diagram of an embodiment of a brainwave regulation device and a control device of the present invention. Referring to FIGS. 1 and 2, a brainwave regulation device 100 of this embodiment is adapted to transmit signals to or receive signals from a control device 200. The brainwave regulation device 100 includes a head wearable element 110, a control module 120, at least one brainwave sensor 130 and at least one ear bone oscillator 140. The control module 120 is disposed on the head wearable element 110 and adapted to transmit signals to or receive signals from the control device 200. The at least one brainwave sensor 130 is disposed on the head wearable element 110 and adapted to transmit signals to or receive signals from the control module 120. The at least one brainwave sensor 130 transmits a detected brainwave signal E1 to the control device 200 through the control module 120. The at least one ear bone oscillator 140 is disposed on the head wearable element 110 and adapted to transmit signals to or receive signals from the control module 120 and receive a control signal C1 transmitted from the control device 200 through the control module 120 to generate oscillation.

In this embodiment, the head wearable element 110 includes, for example, a head band, a hat, an ear hanging element or other elements wearable on head of the user. In this embodiment, the head wearable element 110 is a head band, but it is not limited thereto. The control module 120 is adapted to transmit signals to and receive signals from the control device 200 and is able to process signals. For example, the control module 120 processes the brainwave signal E1 detected by the at least one brainwave sensor 130 and transmits the brainwave signal E1 to the control device 200, and the control module 120 receives the control signal C1, processes the control signal C1 and transmits the control signal C1 to the at least one ear bone oscillator 140. The control device 200 is, for example, a portable electronic device. Precisely, the control device 200 is, for example, a device having a display screen, such as a mobile phone, a smart watch, a laptop or a tablet, to allow a user to view personal information and brain wave information or input information. The control device 200 can also be a non-portable electronic device such as a desktop.

In this embodiment, the amount of the brainwave sensor 130 is one for example, and the amount of the ear bone oscillator 140 is also one for example. According to various requirements, the amount of the brainwave sensor 130 and the ear bone oscillator 140 can also be more than one. The amount of the brainwave sensor 130 and the amount of the ear bone oscillator 140 are equal or not equal. The amount of the brainwave sensor 130 and the amount of the ear bone oscillator 140 are not limited in the present invention.

The ear bone oscillator 140 is disposed on the head wearable element 110. The ear bone oscillator 140 oscillates to induce oscillation of the ear bone. The oscillation is transferred to the internal ear to induce impulses of auditory nerve in the internal ear. The impulses are transferred to auditory center and brain to affect the brainwave frequency of a user, whereby the brainwave frequency is regulated.

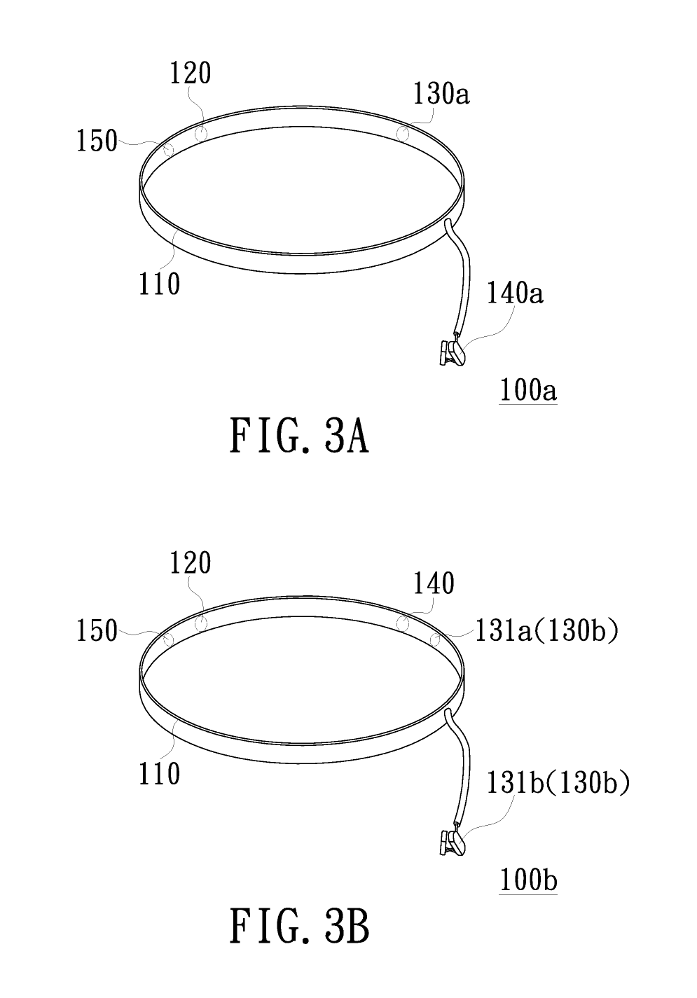

In addition, in another embodiment of the present invention, one of a brainwave sensor 130a and an ear bone oscillator 140a of a brainwave regulation device 100a for example protrudes from the head wearable element 110 and connects to the head wearable element 110. As illustrated in FIG. 3A, in this embodiment, the brainwave sensor 130a is, for example, disposed on the head wearable element 110 and the ear bone oscillator 140a is connected to the head wearable element 11, but the present invention is not limited thereto.

In addition, as illustrated in FIG. 3B, in another embodiment of the present invention, a brainwave regulation device 100b includes, for example, a plurality of brainwave sensors 130b. In this embodiment, the brainwave regulation device 100b includes two brainwave sensors 131a and 131b, but the present invention is not limited thereto. In this embodiment, the brainwave sensor 131a is disposed on the head wearable element 110, and the brainwave sensor 131b is connected to the head wearable element 110 to detect brainwave of the user.

Referring to FIGS. 1 and 2 again, the control device 200 of this embodiment includes, for example, a transmission element corresponding to the control module 120 so that the control device 200 and the control module 120 are capable of transmitting signals to each other. The signal transmitted between the control device 200 and the control module 120 includes a blue tooth signals, wireless network signal, a near field communication signals, a Zigbee signal, an ultra-wideband signal, or a light fidelity signal. The present invention does not limit the type of the signal transmitted between the control device 200 and the control module 120.

The control device 200 further includes, for example, software such as mobile application configured to calculate the received brainwave signal E1 to generate the control signal C1. The control signal C1 is then transmitted to the control module 120 by the control device 200.

As illustrated in FIG. 2, when the user regulates brainwave frequency utilizing the brainwave regulation device 100, the head wearable element 110 is placed on the user's head, and the brainwave sensor 130 detects the user's brainwave frequency and generates the brainwave signal E1. The brainwave signal E1 is transmitted to the control device 200 by the control module 120 disposed in the head wearable element 110. The control device 200 generates the control signal C1 and transmits the control signal C1 to the control module 120. The control signal C1 is further transmitted to the ear bone oscillator 140 for oscillation. The user receives the oscillation of the ear bone oscillator 140, and the ear bone of the user is induced to oscillate so as to regulate the user's brainwave frequency and provide a comfortable sleep or good spirit. The method of generating the control signal C1 according to the brainwave signal E1 is described in details in the following paragraphs.

Since the brainwave sensor 130 and the ear bone oscillator 140 are provided on the head wearable element 110, the brainwave regulation device 100 of this embodiment allows the user to detect the brainwave frequency by the brainwave sensor 130 and generate vibration by the ear bone oscillator 140 to adjust the user's brainwave frequency, thereby achieving the effect of having a comfortable sleep and good spirit.

In addition, the brainwave regulation device 100 further includes, for example, an identification element 150 disposed on the head wearable element 110. The identification element 150 is, for example, an induction identification element so that the control device 200 is capable of detecting the identification element 150 and shows information of a user. The identification element 150 includes radio frequency element or identification code. Precisely, the radio frequency element, for example, includes radio frequency identification (RFID) chips, and the identification code is, for example, a barcode including one-dimensional barcode such as linear barcode, two-dimensional barcode such as quick response code (QR code) or three-dimensional barcode such as Colormobi. In addition, in other embodiments, for example, the control module 120 includes a software identification chips. The software identification chip includes codes which are burned in the software identification chip to allow a user to identify the codes in the software identification chip so as to transmit the user's information to the control device 200. However, it is not limited thereto. The user's information includes user's personal information and utilization record. The user can realize his sleep condition and mental condition according to his/her personal utilization record. However, it is not limited thereto.

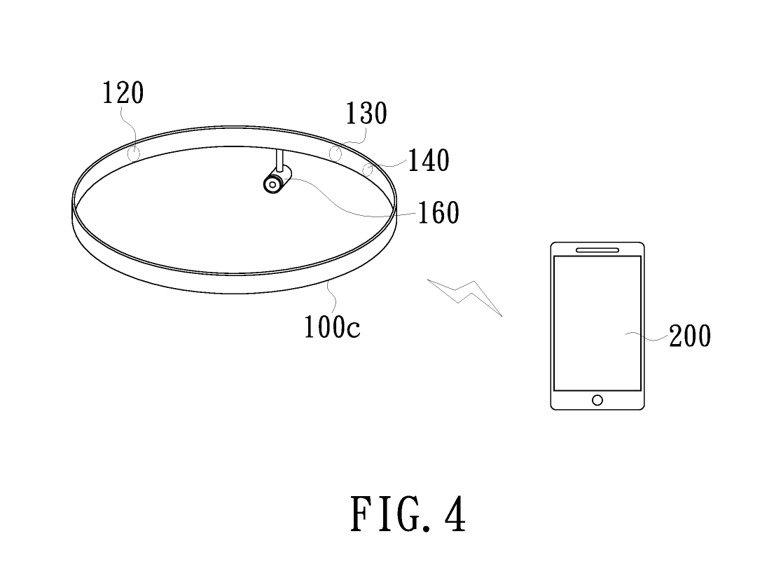

As illustrated in FIG. 4, in still another embodiment of the present invention, the brainwave regulation device 100c further includes, for example, an image capturing device 160 for replacing the identification element 150. The image capturing device 160 is adapted to capture images of a user's face or iris, and the captured images are transmitted to the control device 200 through the control module 120. The control device 200 recognizes the user based on the captured images and displays personal information stored in the control device 200 such as the user's personal data and utilization record. In addition, in this embodiment, the control device 200 is, for example, a mobile phone, but it is not limited thereto.

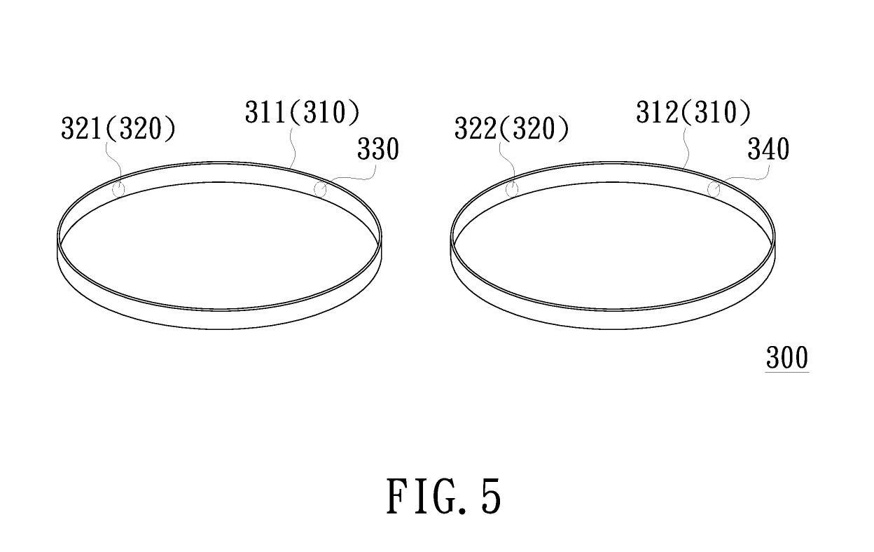

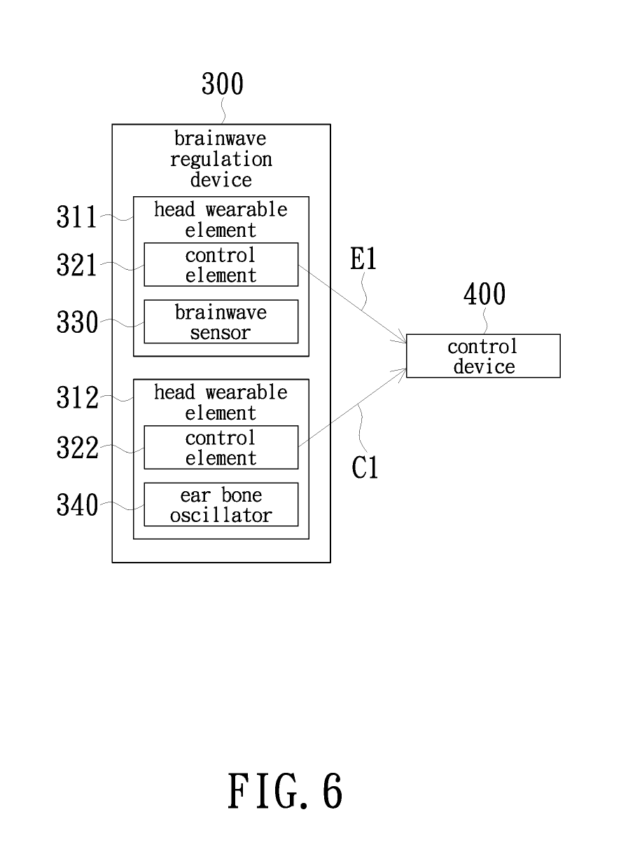

FIG. 5 is a schematic diagram of another embodiment of a brainwave regulation device of the present invention, and FIG. 6 is a block diagram of another embodiment of a brainwave regulation device and a control device of the present invention. Referring to FIGS. 5 and 6, in another embodiment of the present invention, a head wearable element 310 includes, for example, two head wearable members 311 and 312. A control module 320 includes two transmission elements 321 and 322. The brainwave sensor 330 and the transmission element 321 are disposed on the head wearable member 311, and an ear bone oscillator 340 and the transmission element 322 are disposed on the head wearable member 312.

When the user uses the brainwave regulation device 300, the brainwave sensor 330 disposed on the head wearable member 311 is used to detect the brainwave signals E1, and the brainwave signals E1 is transmitted to the control device 400 through the transmission element 321. Then, the control device 400 disposed on the head wearable member 312 is used to transmit the control signal C1 to the transmission element 322. The control signal C1 is transmitted to the ear bone oscillator 340 disposed on the head wearable member 312 through the transmission element 322 to oscillate.

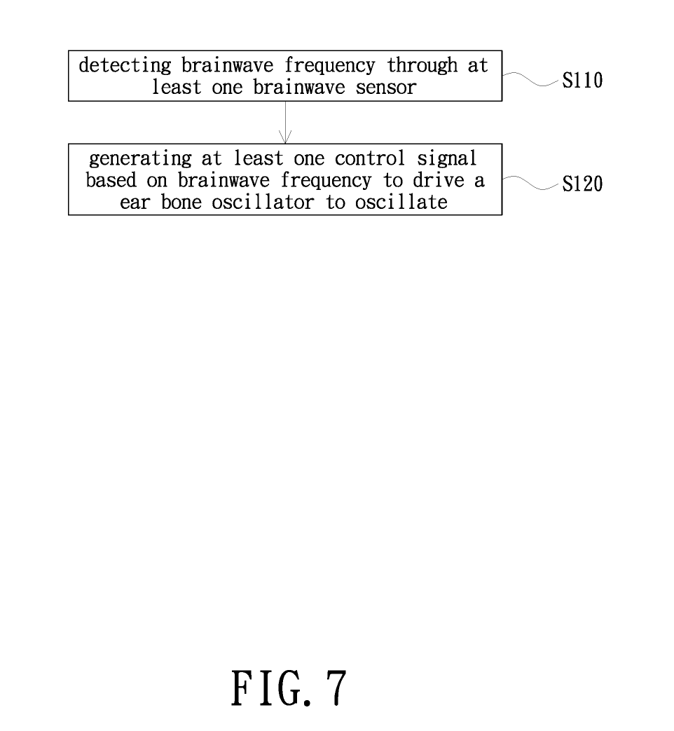

FIG. 7 is a block diagram of an embodiment of a brainwave regulation method of the present invention. Referring to FIG. 7, the brainwave regulation method is adapted to use with the above-described brainwave regulation device to perform a brainwave regulation. In the brainwave regulation method of the present invention, for example, as shown in step S110, it first performs: detecting brainwave frequency through at least one brainwave sensor. In this embodiment, for example, the control device receives the brainwave frequency detected by at least one brainwave sensor.

Afterwards, as shown in step S120, it performs: generating at least one control signal based on brainwave frequency to drive an ear bone oscillator to oscillate. In this embodiment, software is installed in the control device, and the software calculates the brainwave frequency to generate at least one control signal. The at least one control signal includes, for example, an oscillating frequency signal and a time signal, and the ear bone oscillator oscillates according to the oscillating frequency signal and time signal of the control signal. Time signal is, for example, one minute, five minutes or ten minutes based on various requirements, but the oscillating time of the ear bone oscillator is not limited.

The method of generating oscillating frequency first, for example, compares the difference between the brainwave frequency and a target frequency and generates the oscillating frequency through a calculation. The oscillating frequency generated by the calculation is, for example, between the brainwave frequency and the target frequency or the target frequency is between the brainwave frequency and the oscillating frequency, so that the user's brainwave frequency is regulated to approach the target frequency through the brainwave regulation method of the present invention and change the user's state. When the oscillating frequency is lower than the brainwave frequency, the user is regulated from an awaken state to a relaxed state or from a relaxed state to a deep sleep state. The awaken state is a state in which the user concentrates on something or thinks something. The relaxed state is a state in which the user has clear consciousness but physical relax. The deep sleep state is a state in which the user is in deep sleep. On the other hand, when the oscillating frequency is higher than the brainwave frequency, the user is regulated from a deep sleep state to a relaxed state or from a relaxed state to an awaken state.

The target frequency is, for example, preset in the software installed in the control device to allow the user to select a desired target frequency. The target frequency ranges, for example, from 0.1 Hz to 100 Hz. For example, when the target frequency ranges from 0.1 Hz to 15 Hz (a wave), the user is regulated to a deep sleep state and may have a comfortable sleep. When the target frequency ranges from 12.5 Hz to 18 Hz (β wave), the user is regulated to a relaxed state. When the target frequency ranges from 25 Hz to 100 Hz (y wave), the user is regulated to an awaken state and may have a good spirit.

During the calculation, the target frequency is G, the brainwave frequency is D, the oscillating frequency is S, and |G-S|≤|D-G|, |G-S|=(1/n)|D-G|, wherein n is greater than 1 and n is, for example, a constant, a linear function or a non-linear function. The user is capable of regulating the difference between the oscillating frequency and the target frequency based on the difference between the brainwave frequency and the target frequency, and further regulating the generated oscillating frequency.

Then, the detection of the brainwave frequency through the brainwave sensor can be repeated according to requirements and the regulation of brainwave can also be performed several times according to the steps of generating control signals from the brainwave frequency. Precisely, when the user has regulated his/her brainwave for a period, the brainwave sensor detects the user's brainwave again, and the brainwave frequency and the target frequency are compared to obtain the difference between the brainwave frequency and the target frequency. Afterwards, the oscillating frequency is generated between the brainwave frequency and the target frequency through the equation |G-S|=(1/n)|D-G| so as to regulate the user's brainwave and enable the user's brainwave frequency more closed to the target frequency.

In the brainwave regulation method of this embodiment, since the ear bone oscillator can oscillate at an appropriate frequency to further regulate the user's brainwave frequency, the user may have a comfortable sleep and good spirit without regulating music or songs to which the user listens.

In summary, the brainwave regulation device of the present invention oscillates the user's ear bone through the era bone oscillator disposed on the head wearable element to further regulate the user's brainwave frequency. The user may have a comfortable sleep or good spirit without wearing headsets. In addition, the brainwave regulation method of the invention generates appropriate oscillating frequency and creates vibration through the ear bone oscillator to further regulate the user's brainwave frequency. Therefore, the user may have a comfortable sleep and good spirit without adjusting the music or songs to which the user is listening.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

### INDUSTRY UTILITY

The brainwave regulation device of the present invention oscillates the user's ear bone through the era bone oscillator disposed on the head wearable element to further regulate the user's brainwave frequency. The user may have a comfortable sleep or good spirit without wearing headsets. In addition, the brainwave regulation method of the invention generates appropriate oscillating frequency and creates vibration through the ear bone oscillator to further regulate the user's brainwave frequency. Therefore, the user may have a comfortable sleep and good spirit without adjusting the music or songs to which the user is listening.

1. A brainwave regulation device adapted to transmit signals to or receive signals from a control device, the brainwave regulation device comprising:
a head-wearable element;
a control module, disposed on the head-wearable element and adapted to transmit signals to or receive signals from the control device;
at least one brainwave sensor, connected to the head-wearable element and adapted to transmit signals to or receive signals from the control module and transmit a detected brainwave signal to the control device through the control module; and
at least one ear bone oscillator, connected to the head-wearable element and adapted to transmit signals to or receive signals from the control module and receives control signals from the control device through the control module to generate oscillation.

2. The brainwave regulation device according to claim 1, further comprising an identification element disposed on the head-wearable element.

3. The brainwave regulation device according to claim 2, wherein the identification element comprises a radio-frequency element or an identification code.

4. The brainwave regulation device according to claim 1, further comprising an image capturing device adapted to capture an image of a face or an iris of a user and transmit the image to the control device through the control module.

5. The brainwave regulation device according to claim 1, wherein the signals transmitted between the control device and the control module comprise a blue tooth signal, a wireless network signal, a near field communication signal, a ZigBee signal, an ultra wideband signal or a light fidelity signal.

6. The brainwave regulation device according to claim 1, wherein the head-wearable element comprises two head wearable members, the control module comprises two control elements, wherein the at least one brainwave sensor and one of the control elements are disposed on one of the head wearable member; the at least one ear bone oscillator and the other one of the control elements are disposed on the other one of the head wearable members.

7. A brainwave regulation method adapted for use with a brainwave regulation device for brainwave regulation, wherein the brainwave regulation device comprises a head wearable element, a control module, at least one brainwave sensor and at least one era bone oscillator, and the brainwave regulation method comprises:
detecting a brainwave frequency through the at least one brainwave sensor; and
generating at least one control signal to drive the era bone oscillator to oscillate, wherein the at least one control signal comprises an oscillating frequency signal and a time signal.

8. The brainwave regulation method according to claim 7, wherein a method of generating the oscillating frequency comprises:
comparing difference between the brainwave frequency and a target frequency to generate an oscillating frequency so as to regulate the brainwave frequency to approach the target frequency and vary a state of a user, wherein when the oscillating frequency is lower than the brainwave frequency, the user is regulated from an awaken state to a relaxed state or from a relaxed state to a deep sleep state; when the oscillating frequency is higher than the brainwave frequency, the user is regulated from a deep sleep state to a relaxed state or from a relaxed state to an awaken state.

9. The brainwave regulation method according to claim 8, wherein the target frequency ranges from 0.1 Hz to 100 Hz.

10. The brainwave regulation method according to claim 8, wherein the target frequency is represented by G, the brainwave frequency is represented by D, the oscillating frequency is represented by S, and |G-S|≤|D-G|, |G-S|=(1/n)|D-G|, wherein n is greater than 1 and n comprises a constant, a linear function or a non-linear function.
